(19) United States
(12) Patent Application Publication
(10) Pub. No.: US 2024/0390477 A1
(43) Pub. Date: Nov. 28, 2024

(54) J PARAMYXOVIRUS VACCINES

(71) Applicant: UNIVERSITY OF GEORGIA RESEARCH FOUNDATION, INC., Athens, GA (US)

(72) Inventor: Biao He, Watkinsville, GA (US)

(21) Appl. No.: 18/686,149

(22) PCT Filed: Aug. 22, 2022
(86) PCT No.: PCT/US2022/041108

§ 371 Date: Feb. 23, 2024

Related U.S. Application Data
(60) Provisional application No. 63/237,374, filed on Aug. 26, 2021.

(51) Int. Cl. A61K 39/145; A61K 39/00; A61P 31/16; C12N 15/86

(52) U.S. Cl. CPC A61K 39/145; A61P 31/16; C12N 15/86; A61K 2039/5256; A61K 2039/543; C12N 2760/16134; C12N 2760/18034; C12N 2760/18043

(57) ABSTRACT

The present invention provides safe, stable, efficacious, and cost-effective vaccines based on viral expression vectors that include a J Paramyxovirus (JPV) genome including a heterologous nucleotide sequence expressing a heterologous polypeptide inserted within the JPV genome.

### CONTINUING APPLICATION DATA

This application claims the benefit of U.S. Provisional Application Ser. No. 63/237,374, filed Aug. 26, 2021, which is incorporated by reference herein.

### GOVERNMENT FUNDING

This invention was made with government support under grant number RO1 AI128924 awarded by the NIH. The government has certain rights in the invention.

### BACKGROUND

Inactivated influenza vaccines have been available since the 1940's and are 60-80% effective against matched influenza virus strains but are less effective against antigenic drift variants and are ineffective against different subtypes. Thus, annual vaccination is needed to prevent infections from new strains or subtypes. Current seasonal influenza vaccines consist of two influenza A viruses (H1N1 and H3N2) and one or two influenza B virus. Moreover, vaccination coverage and production continue to be problems worldwide. Current licensed influenza virus vaccines are produced in chicken eggs, requiring the availability of millions of eggs and significant time between identification of vaccine strains and availability of vaccines. Additionally, this vaccination strategy provides no protection against unexpected strains, outbreaks, or pandemics. New vaccination strategies are needed for the prevention and control of influenza virus infection.

### SUMMARY OF THE INVENTION

The present disclosure includes a viral expression vector comprising a J Paramyxovirus (JPV) genome comprising a heterologous nucleotide sequence expressing a heterologous polypeptide inserted within the JPV genome.

In some aspects, at least a portion of a JPV gene has been replaced with the heterologous nucleotide sequence.

In some aspects, the heterologous nucleotide sequence replaces at least a part of the N gene of the JPV genome; the P gene of the JPV genome; the M gene of the JPV genome; the F gene of the JPV genome; the SH gene of the JPV genome; the TM gene of the JPV genome; the G gene of the JPV genome; the X gene of the JPV genome; and/or the L gene of the JPV genome.

In some aspects, the heterologous nucleotide sequence replaces the SH gene nucleotide sequence.

In some aspects, the heterologous nucleotide sequence is inserted between the N and P genes of the JPV genome; between the P and M genes of the JPV genome; between the M and F genes of the JPV genome; between the F and SH genes of the JPV genome; between the SH and TM genes of the JPV genome; between the TM and G genes of the JPV genome; between the G and X genes of the JPV genome; and/or between the X and L genes of the JPV genome.

In some aspects, wherein the heterologous nucleotide sequence is inserted within the N gene of the JPV genome; within the P gene of the JPV genome; withing the M gene of the JPV genome; within the F gene of the JPV genome; within the SH gene of the JPV genome; within the TM gene of the JPV genome; within the G gene of the JPV genome; within the X gene of the JPV genome; and/or within the L gene of the JPV genome.

In some aspects, the JPV genome further comprises one or more mutations.

In some aspects, the heterologous polypeptide comprises an influenza hemagglutinin (HA), an influenza neuraminidase (NA), an influenza nucleocapsid protein (NP), influenza M1, influenza M2, influenza PA, influenza PB1, influenza PB2, influenza PB1-F2, influenza NS1 or influenza NS2. In some aspects, the influenza comprises influenza A, influenza B, or influenza C virus. In some aspects, the heterologous polypeptide comprises a hemagglutinin (HA) from influenza A virus strain subtype H1 to H18. In some aspects, the heterologous polypeptide comprises a hemagglutinin (HA) from influenza A virus strain H5N1, H3N2, or H1N1. In some aspects, the heterologous polypeptide comprises a hemagglutinin (HA) from influenza A virus strain H5N1. In some aspects, the heterologous polypeptide comprises a hemagglutinin (HA) from influenza A virus strain H5N1 and the heterologous nucleotide sequence replaces the SH gene nucleotide sequence. In some aspects, the heterologous polypeptide comprises an influenza neuraminidase (NA) from influenza type A subtype N1 to N10. In some aspects, the NP, M1, M2, PA, PB1, PB2, PB1-F2, NS1 or NS2 is from influenza A virus strain H1 to H17 and the NA is from influenza A virus strain from N1 to N10.

In some aspects, the heterologous polypeptide is derived from human immunodeficiency virus (HIV), parainfluenza virus 1, parainfluenza virus 2, parainfluenza virus 3, parainfluenza virus 4, human respiratory syncytial virus, bovine respiratory syncytial virus, human metapneumovirus, avian influenza, canine influenza, avian metapneumovirus, Nipah virus, Hendra virus, rabies virus, Ebola virus, porcine circovirus, porcine reproductive and respiratory syndrome virus, swine influenza virus, New Castle disease virus, mumps virus, measles virus, canine distemper virus, feline leukemia virus, human calicivirus, veterinary calicivirus, human norovirus, veterinary norovirus, rinderpest virus, Mycobacterium tuberculosis, and/or an emerging influenza virus in humans or animals.

In some aspects, the heterologous polypeptide is derived from a bacterium or a parasite.

In some aspects, the viral expression vector comprises two or more heterologous nucleotide sequence expressing a heterologous polypeptide.

The present disclosure includes a viral particle comprising a viral expression vector as described herein.

The present disclosure includes a composition of the viral expression vector or viral particle as described herein. In some aspects, the composition further comprises an adjuvant.

The present disclosure includes a method of expressing a heterologous polypeptide in a cell, the method comprising contacting the cell with a viral expression vector, viral particle, or composition as described herein.

The present disclosure includes a method of inducing an immune response in a subject to a heterologous polypeptide, the method comprising administering a viral expression vector, viral particle, or composition as described herein to the subject. In some aspects, the immune response comprises a humoral immune response and/or a cellular immune response. In some aspects, the viral expression vector, viral particle, or composition is administered intranasally, intramuscularly, topically, orally, or in ovo.

The present disclosure includes a method of expressing a heterologous polypeptide in a subject, the method comprising administering a viral expression vector, viral particle, or composition as described herein to the subject. In some aspects, the viral expression vector, viral particle, or composition is administered intranasally, intramuscularly, topically, orally, or in ovo.

The present disclosure includes a method of vaccinating a subject, the method comprising administering a viral expression vector, viral particle, or composition as described herein to the subject. In some aspects, the viral expression vector, viral particle, or composition is administered intranasally, intramuscularly, topically, orally, or in ovo.

### BRIEF DESCRIPTION OF THE FIGURES

FIGS. 1A and 1B. Recovery of recombinant virus rJPV-ASH-H5. FIG. 1A presents schematics of rJPV and rJPV-ASH-H5, indicating the location where the ORF of SH was replaced with HA of H5N1. To confirm the presence of H5 HA in rJPV-ASH-H5, RNA was extracted from the media of infected cells, and RT-PCR was performed using primers MA12F and MA09R to amplify the insertion region (FIG. 1). Expected sizes of the PCR products are 3185 bp (rJPV-ASH-H5) and 1709 bp (rJPV).

FIGS. 2A-2C. Gene expression of rJPV and rJPV-ΔSH-H5 in vitro. Immunofluorescent staining of Vero cells infected with rJPV or rJPV-ΔSH-H5 is shown in FIG. 2A. Vero cells were mock-infected or infected with rJPV or JPV-ΔSH-H5. At 2 d.p.i, cells were washed with PBS and fixed with 0.5% formaldehyde. The cells were permeabilized with 0.1% PBS-saponin solution incubated for 30 min with polyclonal anti-JPV-F or -H5N1 HA monoclonal antibody at a 1:100 dilution, and FITC-labelled with goat anti-mouse antibody. The cells were incubated for 30 min and examined and photographed using a Nikon FXA fluorescence microscope. Expression of H5N1 HA in virus-infected cells is shown in FIG. 2B. Vero cells in the six-well plates were mock-infected or infected with rJPV or rJPV-ΔSH-H5 at an MOI of 5. The cells were collected at 2 d.p.i and fixed with 0.5% formaldehyde for 1 h. The fixed cells were resuspended in FBS-DMEM (50:50) and permeabilized with 70% ethanol overnight. The cells were washed once with PBS and then incubated with mouse anti-HA monoclonal antibody. Secondary staining was performed using APC Goat anti-mouse IgG, and the fluorescence intensity was measured with a flow cytometer. Samples are triplicates, and error bars show standard errors of the means. Comparison of rJPV and rJPV-ΔSH-H5 growth in vitro is shown in FIG. 2C. Low MOI growth curve of rJPV and rJPV-ΔSH-H5. Vero cells in a 6 well plate were infected, in triplicates, with rJPV or rJPV-ΔSH-H5 at an MOI of 0.1, and the medium was harvested at 24-h intervals. Plaque assays were performed on Vero cells to determine the virus titer. Statistical significance between groups at each time point was calculated based on two-way ANOVA to compare the growth kinetics (P<0.001***, P<0.01**, P<0.05*).

FIGS. 3A-3C. Immunization of BALB/c mice with PBS, rJPV-ΔSH-H5, and rPIV5-H5. In FIG. 3A, BALB/c mice were intranasally infected with 100 μl of PBS, rJPV-ΔSH-H5, or rPIV5-H5 at a dose of 1×10⁵ PFU (n=10 per group). Mice were monitored daily, and weight loss was graphed as the average percentage of their original weight (on the day of infection). Anti-H5 IgG titers of immunized mice are shown in FIG. 3B. BALB/c mice were intranasally immunized with PBS or rJPV-ΔSH-H5 or rPIV5-H5 at 10⁵ PFU and bled on day 28 post-immunization. The mouse blood samples were collected for analysis. Purified recombinant HA protein was used to coat the ELISA plates. OD₄₅₀ values were measured using a plate reader. Antibody titer was defined as the highest serum dilution at which the OD₄₅₀ was higher than the PBS average OD₄₅₀ plus two times the standard deviation. PR8-H5N1 neutralization by rPIV5-H5- and rJPV-ΔSH-H5-immunized Balb/C mouse serum is shown in FIG. 3C. Serum was serially diluted and incubated 1:1 with 50 PFU PR8-H5N1 before being added to MDCK cells. Agarose overlays were added, and plaques were counted five days later. The neutralization titer of each sample was defined as the highest dilution at which the plaque reduction was 50% or less. Error bars represent the standard error of the means, and statistical significance was calculated with one-way ANOVA (****, p<0.0001; ***, p<0.001; **, p<0.01; * p<0.05).

FIGS. 4A and 4B. Efficacy of rJPV-ΔSH-H5 against HPAI H5N1 challenge in mice. The mice were inoculated with PBS, rJPV-ΔSH-H5, or rPIV5-H5 (n=10 per group) at a dose of 10⁵ PFU per mouse. At 73 dpi, the mice were challenged with HPAI H5N1 at a dose of 10 LD50. Weights of mice challenged with H5N1 are shown in FIG. 4A. Weights were monitored daily after the challenge for ten days. Weight is graphed as the average percentage of original weight (the day of the challenge). Error bars represent the standard error of the mean. Survival rates are shown in FIG. 4B.

FIG. 5. Immunization of rhesus macaques with rJPV-ΔSH-H5. Four rhesus macaques were intranasally immunized with 2.1×10⁶ PFU of rJPV-ΔSH-H5 at week 0, week 4, and week 12, respectively. Animals were monitored daily, and clinical samples (blood, nasal swabs, and rectal fluids) were collected regularly for ELISA, ELISpot, ICS, neutralization assay, and virus plaque assay.

FIGS. 6A-6C. Antibody-secreting cell responses in PBMCs after immunization of NHPs with rJPV-ΔSH-H5. In FIG. 6A the total numbers of plasmablasts secreting IgG or IgA in peripheral blood were quantified by ELISpot assay on baseline and on day 5 following each immunization as a measure of overall plasmablasts stimulation. Total antibody-secreting cells were enumerated and expressed as spots per million PBMCs. In FIG. 6B H5 HA-specific plasmablasts secreting IgG or IgA in peripheral blood were quantified by ELISpot at the same time at the total ones and enumerated as HA-specific IgG and IgA-secreting cells per million PBMCs. In FIG. 6C the percentage of antigen-specific IgG (filled symbol) and IgA (open symbol) frequencies per total plasmablasts was derived as a measure of antigenic stimulation. Each solid black arrow represents rJPV-ΔSH-H5 intranasal administration. Error bars indicate the standard errors of the means. Statistical analyses were considered significant of P values as follows: * P<0.05; n.s, P>0.05, not significant.

FIGS. 7A-7E. Antibody responses in bone marrow plasma cells and sera after immunization of NHPs with rJPV-ΔSH-H5. For FIG. 7A total plasma cells were quantified in bone marrow by ELISpot assay at baseline and at 2 weeks after each booster immunization. Total antibody-secreting cells were enumerated and expressed as spots per million plasma cells. In FIG. 7B H5 HA-specific plasma cells in bone marrow were quantified by ELISpot assay at the same time points expressed as spots per million bone marrow cells, as well as the percentage of antigen-specific IgG and IgA frequencies relative to the total IgG and IgA producing plasma cells in bone marrow (FIG. 7C). Each solid black arrow represents rJPV-ΔSH-H5 intranasal administration. Error bars indicate the standard errors of the means. Statistical analyses were considered significant of P values as follows: * P<0.05; n.s, P>0.05, not significant. For FIG. 7D, anti-H5 HA IgG titers were measured via ELISA and plotted for each animal. The antibody titer for each sample was defined as the highest serum dilution at which the OD₄₅₀ was higher than the week 0 average OD₄₅₀ plus two times the standard deviation. FIG. 7E shows PR8-H5N1 neutralization by rJPV-ΔSH-H5-immunized monkey serum. Serum was serially diluted and incubated 1:1 with 50 PFU PR8-H5N1 before being added to MDCK cells. Agarose overlays were added, and plaques were counted five days later. The neutralization titer of each sample was defined as the highest dilution at which the plaque reduction was 50% or less.

FIGS. 8A-8D. Cell-mediated responses of immunization of NHPs with rJPV-ΔSH-H5. Induction of HA-specific CD4+(FIG. 8A) and CD8+(FIG. 8B) T cells responses following prime and boost regimen. Each solid black arrow represents rJPV-ΔSH-H5 intranasal administrations. PBMC samples were restimulated in vitro with HA peptides for 6 hours and tested for the production of IFN-γ, TNF-α, IL-17A, MIP-1β and CD107a. In FIG. 8C, overall T cell responses to the immunizations are represented by the sum of all individual cytokines responses to HA peptide pools for CD4 and CD8 T cells. FIG. 8D shows HA-specific CD4 and CD8 T cell polyfunctional responses at weeks 2, 6, and 14, respectively. Pie charts represent the distribution of T cell cytokine response profiles as mono-, 2-, 3-, 4-, or 5-functions. Analyses were performed with the Pestle and SPICE software. Student's t-test and permutation test were used for pie comparison between two time points. The level of significance is indicated by P-values as follows: * P<0.05; n.s, P>0.05, not significant.

### DETAILED DESCRIPTION

J Paramyxovirus (JPV) is a non-segmented negative-strand RNA virus and a member of the proposed genus Jeilongvirus in the family Paramyxoviridae. The present invention provides engineered constructs of the JPV genome that include one or more heterologous nucleotide sequences expressing one or more heterologous polypeptides inserted within the JPV genome. Such JPV constructs can serve as viral expression vectors, including for use as improved vaccine vectors.

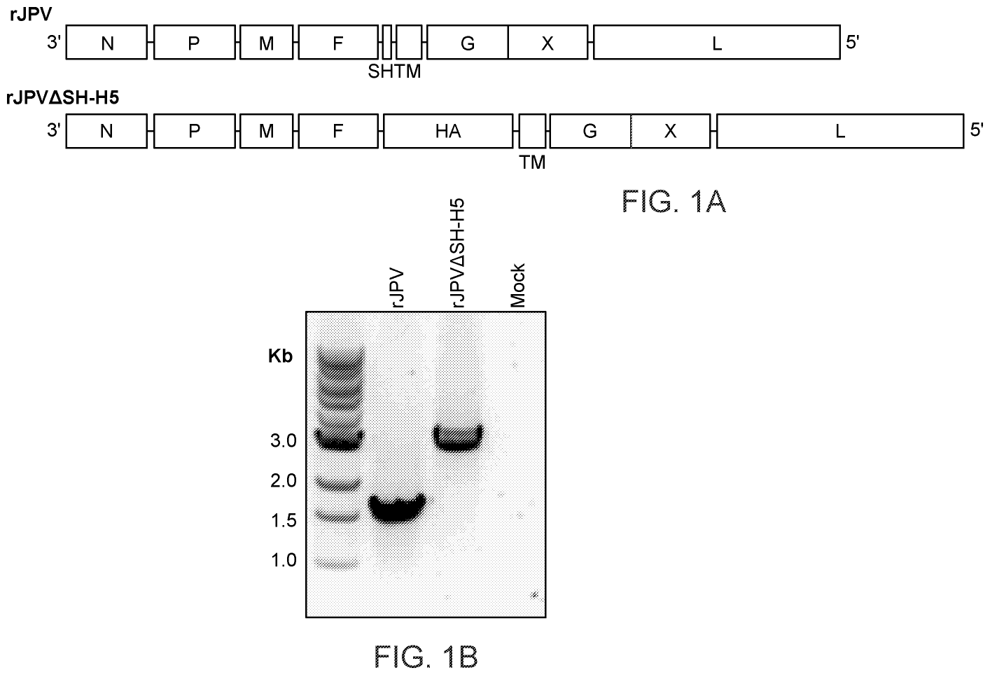

JPV was isolated from moribund mice with hemorrhagic lung lesions in the early 1970s in Australia (Jun et al., 1977, Aust J Exp Biol Med Sci; 55:645-647). The JPV genome structure was determined in 2005, and it has eight genes in the order of 3′-N-P/V/C-M-F-SH-TM-G-L-5′ (Jun et al., 1977, Aust J Exp Biol Med Sci; 55:645-647; and Jack et al., 2005, J Virol; 79:10690-10700). FIG. 1A shows the JPV genome structure. JPV has a large genome size of 18,954 nucleotides and includes several genes that distinguish Jeilongviruses from other paramyxoviruses. The transmembrane (TM) gene is located between the small hydrophobic (SH) and glycoprotein (G) genes and is only found in members of the Jeilongvirus genus. Along with the fusion (F) and G proteins, TM promotes cell-to-cell fusion. However, TM is not necessary for virus-to-cell fusion, and a recombinant JPV virus lacking TM can be recovered and grown to similar titers as wild-type (WT) JPV (Li et al., 2015, Proc Natl Acad Sci USA; 112:12504-12509). Further separating JPV from other Paramyxoviruses and Jeilongviruses, the JPV G gene is significantly larger than other Paramyxovirus G genes and includes a 2115 nucleotide second open reading frame (ORF) immediately following the G ORF stop codon. This open reading frame, named ORF-X, is in frame with G, and its first methionine is the 30^{th} amino acid, suggesting that there is a potential G-X intergenic region suitable for binding of the polymerase (Jack et al., 2005, J Virol; 79:10690-10700) Currently, the role of X is unknown. TM is unique to Jeilongviruses and JPV, is not essential, and can likely be replaced with foreign antigens to generate new viral vectors. Lastly, JPV has a small hydrophobic (SH) gene that is not found in all Paramyxoviruses. JPV SH inhibits TNF-α production and viral-induced apoptosis. Deleting SH attenuates the virus in vivo but does not affect its growth or protein production in vitro (Abraham et al., 2018, J Virol; 92: e00653-18).

Non-segmented negative-sense single-stranded viruses (NNSVs) such as JPV stably express foreign genes without integrating into the host genome. JPV-specific antibodies have been detected in in numerous animals, such as rodents, bats, pigs, and humans, indicating that JPV has a large host range and zoonotic potential (Li et al., 2005, Science; 310:676-679). However, the virus is not associated with disease in any species other than mice. JPV replicates in the respiratory tract of mice and efficiently expresses the virus-vectored foreign genes in tissue culture cells. These characteristics make JPV a safe choice for engineering viral-vectored vaccines. Recently, a Vesicular stomatitis virus (VSV)-vectored Ebola vaccine was approved for human use (Callaway, 2019, Nature; 575:425-426). Parainfluenza virus 5 (PIV5) is a member of the Rubulavirus genus of the family Paramyxoviridae, which is used as a vector for vaccine development against many bacterial and viral diseases (Chen et al., 2015, Vaccine; 33:7217-7224; and Phan et al., 2014, Vaccine; 32:3050-3057). For example, recombinant PIV5 expressing HA of H5N1 was efficacious in protecting mice against HPAI H5N1 challenge at very low doses (Li et al., 2013, J Virol; 87:354-62; Mooney et al., 2013, J Virol; 87:363-71; and Li et al., 2015, PLoS One; 10:e0120355).

Disclosed herein are engineered constructs of the JPV genome that include one or more heterologous nucleotide sequences inserted within the JPV genome, wherein the one or more heterologous nucleotide sequences encode and express one or more heterologous polypeptides.

In some embodiments, a heterologous nucleotide sequence expressing a heterologous polypeptide may be inserted to replace all or part of a JPV gene within the JPV genome. For example, a heterologous nucleotide sequence expressing a heterologous polypeptide may replace the N, P, M, F, SH, TM, G, X, or L gene of the JPV genome. In some preferred embodiments, a heterologous nucleotide sequence expressing a heterologous polypeptide may replace all or part of the SH gene.

In some embodiments, a heterologous nucleotide sequence expressing a heterologous polypeptide may be inserted within a JPV gene, resulting in the expression of a chimeric polypeptide. For example, the heterologous nucleotide sequence expressing a heterologous polypeptide may be inserted within the N gene nucleotide sequence, within the P gene nucleotide sequence, within the M gene nucleotide sequence, within the F gene nucleotide sequence, within the SH gene nucleotide sequence, within the G gene nucleotide sequence, within the X gene nucleotide sequence, within the G gene nucleotide sequence, and/or within the L gene nucleotide sequence of a JPV genome.

A heterologous nucleotide sequence expressing a heterologous polypeptide may, for example, be a heterologous DNA or a heterologous RNA. The heterologous polypeptide may be antigenic and have utility as a vaccine. Such an antigenic polypeptide may be from any of a wide variety of pathogens and diseases affecting humans and/or animals. In some aspects, a heterologous polypeptide may be derived, for example, from human immunodeficiency virus (HIV), parainfluenza virus 1, parainfluenza virus 2, parainfluenza virus 3, parainfluenza virus 4, human respiratory syncytial virus, bovine respiratory syncytial virus, human metapneumovirus, Mycobacterium tuberculosis, avian metapneumovirus, T. cruzi, Nipah virus, Hendra virus, rabies virus, Ebola virus, porcine circovirus, porcine reproductive and respiratory syndrome virus, swine influenza virus, New Castle disease virus, mumps virus, measles virus, canine distemper virus, swine influenza, human calicivirus, veterinary calicivirus, human norovirus, veterinary norovirus, rinderpest virus, influenza A virus, influenza B virus, influenza C virus, or an emerging influenza virus in humans and in animals. In some aspects, a heterologous polypeptide may be derived from a bacterium or a parasite. In some aspects, a heterologous polypeptide may be a cancer antigen.

In some aspects, the encoded heterologous polypeptide is from an influenza virus, including, but not limited to, influenza A, influenza B, or influenza C. Influenza is a negative-sense, segmented RNA virus in the family Orthomyxoviridae. Influenza causes 3-5 million severe cases annually, with 250,000-500,000 deaths globally, with thousands of hospitalizations and deaths every year in the United States. It is classified into subtypes based on the major antigenic surface glycoproteins, hemagglutinin (HA) and neuraminidase (NA). Thus far there are 17 different HA subtypes and 9 different NA subtypes, all containing segments of avian origin. Influenza has the capacity to reassort, whereby gene segments are exchanged creating a new influenza virus to which the population is immunologically naive.

Among influenza viruses, Influenza A viruses are responsible for epidemics in humans, swine, horses, as well as devastating outbreaks in poultry (Webster, 1997, Arch Virol; 13:105-13). Migratory waterfowl, including ducks, seabirds, or shorebirds, are the natural hosts of influenza viruses and from where they jump the species barrier and cause disease in humans (Alexander, 2000, Veterinary Microbiology; 74:3-13). H5N1 HPAI is primarily restricted within the poultry species, but it has emerged as a danger for humans by jumping into many mammalian hosts. Since 1997 H5N1 HPAI has been responsible for 600 human infections, with more than 300 deaths reported from broad geographical areas, including Asia, middle-east, and Africa (Van Kerkhove et al., 2011, PLoS One; 6:e14582). Higher mortality rates and considering the possibility of the emergence of more virulent viruses from the avian source, and the ever-present threat of mutations allowing direct human-to-human transmission make H5N1 viruses a significant public health threat. H5N1 HPAI viruses are not easily transmitted among humans or other mammals, but the spread of these viruses into new geographical regions and wild bird hosts may produce multiple clades with increased genetic diversity through genetic reassortment or antigenic drift. Eradication efforts were unsuccessful and led to the emergence of many antiviral resistant strains (Banner and Kelvin, 2012, J Infect Dev Ctries; 6:465-469; Neumann et al., 2010, Cell Res; 20:51-61; and Sambhara and Poland, 2010, Annu Rev Med; 61:187-98). The immunogenicity of the FDA-approved H5N1 vaccine is low compared to the seasonal influenza vaccines. Inactivated virus vaccines given multiple times at a high concentration provide protection of about 50% in clinical trials (Treanor et al., 2006, N Engl J Med; 354:1343-1351).

The reemergence of a pandemic H1N1 strain in 2009 (Neumann et al., 2009, Nature; 459:931-9) and the emergence of HPAI H5N1 and H7N9 influenza viruses (de Jong et al., 1997, Nature; 389:554; and Gao et al., 2013, N Engl J Med; 368:1888-1897) confirms that influenza is a prominent global threat. Although influenza vaccines have been available commercially since the 1940s, there are many limitations to these vaccines regarding availability and effectiveness. Currently most licensed influenza vaccines are produced in chicken eggs, which requires extensive time between the identification of vaccine strains and vaccine availability. Other limitations include lengthy regulatory approval procedures, limited worldwide vaccine availability, limited efficacy in elderly and unprimed populations, and lack of cross-reactivity requiring reimmunization during each season. The HPAI H5N1 virus was isolated for the first time from geese in Guangdong Province, China, in 1996 (Xu et al., 1999, Virology; 261:15-9).

A heterologous polypeptide may be a hemagglutinin (HA), neuraminidase (NA), nucleocapsid protein (NP), M1, M2, PA, PB1, PB2, NS1 or NS2 from an influenza virus. HA, NA, NP, M1, M2, PA, PB1, PB2, NS1, or NS2 may be for example from influenza A, influenza B, or influenza C. HA, NA, NP, M1, M2, PA, PB1, PB2, NS1, or NS2 may be for example from influenza A virus strain H5N1, H3N2, H1N1, or H7N9.

In some embodiments, a heterologous polypeptide may be a hemagglutinin (HA). The HA may be from, for example, influenza A subtype H1, influenza A subtype H2, influenza A subtype H3, influenza A subtype H4, influenza A subtype H5, influenza A subtype H6, influenza A subtype H7, influenza A subtype H8, influenza A subtype H9, influenza A subtype H10, influenza A subtype H11, influenza A subtype H12, influenza A subtype H13, influenza A subtype H14, influenza A subtype H15, or influenza A subtype H16. HA may be, for example, from influenza A virus strain H5N1, H3N2, H1N1, or H7N9. In some aspects, the HA polypeptide may include a mutation to prevent cleavage.

In some embodiments, a heterologous polypeptide may be a hemagglutinin (HA), including, but not limited to HA from influenza A virus strain H5N1, H3N2, H1N1, or H7N9 and the heterologous nucleotide sequence expressing the heterologous polypeptide replaces the SH gene of JPV. As described in the examples included herewith hemagglutinin (HA) of H5N1 was incorporated into the JPV genome by replacing the small hydrophobic (SH) gene to generate a recombinant JPV expressing HA (rJPV-ΔSH-H5). A single intranasal administration of rJPV-ΔSH-H5 protected mice from a lethal HPAI H5N1 challenge. Intranasal vaccination of rJPV-ΔSH-H5 in rhesus macaques elicited antigen-specific humoral and cell-mediated immune responses.

In some embodiments, a heterologous polypeptide may be a neuraminidase (NA). The NA may be from, for example, influenza A subtype N1, influenza A subtype N2, influenza A subtype N3, influenza A subtype N4, influenza A subtype N5, influenza A subtype N6, influenza A subtype N7, influenza A subtype N8, or influenza A subtype N9 of influenza A. NA may be, for example, from influenza A virus strain H5N1, H3N2, H1N1, or H7N9. In some embodiments, a heterologous polypeptide may be a NA and the heterologous nucleotide sequence expressing the heterologous polypeptide replaces the SH gene of JPV.

Rabies virus (RABV) infection leads to rabies in warm-blooded animals including humans characterized with acute encephalitis at early phase and fatality at later stage without post-exposure treatment (Rupprecht et al., 2006, Expert Rev Anti Infect Ther; 4:1021-1038). Untreated rabies virus infection leads to death. Vaccine and post-exposure treatment have been effective in preventing RABV infection. However, due to cost, rabies vaccination and treatment have not been widely used in developing countries. There are 55,000 human deaths caused by rabies annually. Stray dogs, wild carnivores and bats are the natural reservoirs of field rabies virus, and these rabid carriers are public health risk to human and domestic animals. Human rabies occurrence is largely attributed to the bite of stray dogs in the developing countries where vaccination of animals is limited, especially in rural areas. An efficacious and cost effective rabies vaccine is needed. In some embodiments, a heterologous polypeptide may include one or more rabies polypeptides, including, but not limited to the rabies virus G glycoprotein (RABV G). In some embodiments, a heterologous polypeptide may be a rabies virus G glycoprotein (RABV G) and the heterologous nucleotide sequence expressing the heterologous polypeptide replaces the SH gene of JPV.

Mycobacterium tuberculosis, the etiological agent of tuberculosis (TB), is an important human pathogen. Bacillus Calmette-Guérin (BCG), a live, attenuated variant of Mycobacterium bovis, is currently the only available TB vaccine despite its low efficacy against the infectious pulmonary form of the disease in adults. Thus, a more-effective TB vaccine is needed. M. tuberculosis expresses and secretes three closely related mycolyl transferases also known as the antigen 85 (Ag85) protein complex (Ag85A, 85B and 85C). Both Ag85A and 85B have been shown to be potent antigens. In some embodiments, a heterologous polypeptide may include an antigenic polypeptide of M. tuberculosis, such as, for example the M. tuberculosis antigens 85A and/or 85B.

In some embodiments, a heterologous polypeptide may be an antigenic polypeptide of M. tuberculosis, including, for example, the M. tuberculosis antigens 85A and/or 85B and the heterologous nucleotide sequence expressing the heterologous polypeptide replaces the SH gene of JPV.

In some aspects, a J Paramyxovirus (JPV) genome including a heterologous nucleotide sequence expressing a heterologous polypeptide inserted within the JPV genome as described herein may include may be multivalent, expressing heterologous polypeptides from more than one source, for example, from two, three, four, five, six, seven, eight, nine, ten, or more sources.

In some embodiments, the JPV genome further includes one or more mutations.

In some aspects, a J Paramyxovirus (JPV) genome including a heterologous nucleotide sequence expressing a heterologous polypeptide inserted within the JPV genome as described herein may serve as a viral expression vector and may demonstrate efficacy as a vaccine.

Also included in the present invention are virions and infectious viral particles that includes a JPV genome including one or more heterologous nucleotide sequences expressing a heterologous polypeptide as described herein.

Also included in the present invention are compositions including one or more of the viral constructs or virions, as described herein. Such a composition may include a pharmaceutically acceptable carrier. As used, a pharmaceutically acceptable carrier refers to one or more compatible solid or liquid fillers, diluents or encapsulating substances which are suitable for administration to a human or other vertebrate animal. Such a carrier may be pyrogen free. The present invention also includes methods of making and using the viral vectors and compositions described herein.

The compositions of the present disclosure may be formulated in pharmaceutical preparations in a variety of forms adapted to the chosen route of administration. One of skill will understand that the composition will vary depending on mode of administration and dosage unit.

The agents of this invention can be administered in a variety of ways, including, but not limited to, intravenous, topical, oral, intranasal, subcutaneous, intraperitoneal, intramuscular, and intratumor deliver. In some aspects, the agents of the present invention may be formulated for controlled or sustained release. An advantage of intranasal immunization is the potential to induce a mucosal immune response.

Also included in the present invention are methods of making and using JPV viral expression vectors, including, but not limited to any of those described herein.

For example, the present invention includes methods of expressing a heterologous polypeptide in a cell by contacting or infection the cell with a viral expression vector, viral particle, or composition as described herein.

For example, the present invention includes methods of inducing an immune response in a subject to a heterologous polypeptide by administering a viral expression vector, viral particle, or composition as described herein to the subject. The immune response may include a humoral immune response and/or a cellular immune response. The immune response may enhance an innate and/or adaptive immune response.

For example, the present invention includes methods expressing a heterologous polypeptide in a subject by administering a viral expression vector, viral particle, or composition as described herein to the subject.

For example, the present invention includes methods of vaccinating a subject by administering a viral expression vector, viral particle, or composition as described herein to the subject.

With the methods of the present invention, any of a variety of modes of administration may be used. For example, administration may be intravenous, topical, oral, intranasal, subcutaneous, intraperitoneal, intramuscular, intratumor, in ovo, maternally, and the like. In some aspects, administration is to a mucosal surface. A vaccine may be administered by mass administration techniques such as by placing the vaccine in drinking water or by spraying the animals' environment. When administered by injection, the immunogenic composition or vaccine may be administered parenterally. Parenteral administration includes, for example, administration by intravenous, subcutaneous, intramuscular, or intraperitoneal injection.

An agent of the present disclosure may be administered at once or may be divided into a number of multiple doses to be administered at intervals of time. For example, agents of the invention may be administered repeatedly, e.g., at least 2, 3, 4, 5, 6, 7, 8, or more times, or may be administered by continuous infusion. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that any concentration ranges set forth herein are exemplary only and are not intended to limit the scope or practice of the claimed compositions and methods.

In some therapeutic embodiments, an “effective amount” of an agent is an amount that results in a reduction of at least one pathological parameter. Thus, for example, in some aspects of the present disclosure, an effective amount is an amount that is effective to achieve a reduction of at least about 10%, at least about 15%, at least about 20%, or at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, compared to the expected reduction in the parameter in an individual not treated with the agent.

As used herein, the term “subject” represents an organism, including, for example, a mammal. A mammal includes, but is not limited to, a human, a non-human primate, and other non-human vertebrates. A subject may be an “individual,” “patient,” or “host.” Non-human vertebrates include livestock animals (such as, but not limited to, a cow, a horse, a goat, and a pig), a domestic pet or companion animal, such as, but not limited to, a dog or a cat, and laboratory animals. Non-human subjects also include non-human primates as well as rodents, such as, but not limited to, a rat or a mouse. Non-human subjects also include, without limitation, poultry, horses, cows, pigs, goats, dogs, cats, guinea pigs, hamsters, mink, and rabbits.

As used herein, “isolated” refers to material removed from its original environment (e.g., the natural environment if it is naturally occurring), and thus is altered “by the hand of man” from its natural state.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

In several places throughout the application, guidance is provided through lists of examples, which examples can be used in various combinations. In each instance, the recited list serves only as a representative group and should not be interpreted as an exclusive list. It is to be understood that the particular examples, materials, amounts, and procedures are to be interpreted broadly in accordance with the scope and spirit of the invention as set forth herein.

All headings throughout are for the convenience of the reader and should not be used to limit the meaning of the text that follows the heading, unless so specified.

The invention is defined in the claims. However, below there is provided a non-exhaustive listing of non-limiting exemplary aspects. Any one or more of the features of these aspects may be combined with any one or more features of another example, embodiment, or aspect described herein. Exemplary Embodiments of the present invention include, but are not limited to, the following.

1. A viral expression vector comprising a J Paramyxovirus (JPV) genome comprising a heterologous nucleotide sequence expressing a heterologous polypeptide inserted within the JPV genome
2. The viral expression vector of Embodiment 1, wherein at least a portion of a JPV gene has been replaced with the heterologous nucleotide sequence.
3. The viral expression vector of Embodiment 1, wherein the heterologous nucleotide sequence replaces a least a portion of:
the N gene of the JPV genome;
the P gene of the JPV genome;
the M gene of the JPV genome;
the F gene of the JPV genome;
the SH gene of the JPV genome;
the TM gene of the JPV genome;
the G gene of the JPV genome;
the X gene of the JPV genome; and/or the L gene of the JPV genome.
   4. The viral expression vector of Embodiment 1, wherein the heterologous nucleotide sequence replaces the SH gene nucleotide sequence.
   5. The viral expression vector of claim Embodiment 1, wherein the heterologous nucleotide sequence is inserted:
between the N and P genes of the JPV genome;
between the P and M genes of the JPV genome;
between the M and F genes of the JPV genome;
between the F and SH genes of the JPV genome;
between the SH and TM genes of the JPV genome;
between the TM and G genes of the JPV genome;
between the G and X genes of the JPV genome; and/or
between the X and L genes of the JPV genome.
   6. The viral expression vector of Embodiment 1, wherein the heterologous nucleotide sequence is inserted:
within the N gene of the JPV genome;
within the P gene of the JPV genome;
withing the M gene of the JPV genome;
within the F gene of the JPV genome;
within the SH gene of the JPV genome;
within the TM gene of the JPV genome;
within the G gene of the JPV genome;
within the X gene of the JPV genome; and/or
within the L gene of the JPV genome.
   7. The viral expression vector of any one of Embodiments 1 to 6, wherein the JPV genome further comprises one or more mutations.
   8. The viral expression vector of any one of Embodiments 1 to 7, wherein the heterologous polypeptide comprises an influenza hemagglutinin (HA), an influenza neuraminidase (NA), an influenza nucleocapsid protein (NP), influenza M1, influenza M2, influenza PA, influenza PB1, influenza PB2, influenza PB1-F2, influenza NS1 or influenza NS2.
   9. The viral expression vector of Embodiment 8, wherein the influenza comprises influenza A, influenza B, or influenza C virus.
   10. The viral expression vector of Embodiment 8, wherein the heterologous polypeptide comprises a hemagglutinin (HA) from influenza A virus strain subtype H1 to H18
   11. The viral expression vector of Embodiment 8, wherein the heterologous polypeptide comprises a hemagglutinin (HA) from influenza A virus strain H5N1, H3N2, or H1N1.
   12. The viral expression vector of Embodiment 8, wherein the heterologous polypeptide comprises a hemagglutinin (HA) from influenza A virus strain H5N1.
   13. The viral expression vector of Embodiment 4, wherein the heterologous polypeptide comprises a hemagglutinin (HA) from influenza A virus strain H5N1.
   14. The viral expression vector of Embodiment 8, wherein the heterologous polypeptide comprises an influenza neuraminidase (NA) from influenza type A subtype N1 to N10.
   15. The viral expression vector of Embodiment 8, wherein the NP, M1, M2, PA, PB1, PB2, PB1-F2, NS1 or NS2 is from influenza A virus strain H1 to H17 and the NA is from influenza A virus strain from N1 to N10.
   16. The viral expression vector of any one of Embodiments 1 to 7, wherein the heterologous polypeptide is derived from human immunodeficiency virus (HIV), parainfluenza virus 1, parainfluenza virus 2, parainfluenza virus 3, parainfluenza virus 4, human respiratory syncytial virus, bovine respiratory syncytial virus, human metapneumovirus, avian influenza, canine influenza, avian metapneumovirus, Nipah virus, Hendra virus, rabies virus, Ebola virus, porcine circovirus, porcine reproductive and respiratory syndrome virus, swine influenza virus, New Castle disease virus, mumps virus, measles virus, canine distemper virus, feline leukemia virus, human calicivirus, veterinary calicivirus, human norovirus, veterinary norovirus, rinderpest virus, Mycobacterium tuberculosis, and/or an emerging influenza virus in humans or animals.
   17. The viral expression vector of any one of Embodiments 1 to 7, wherein the heterologous polypeptide is derived from a bacterium or a parasite.
   18. The viral expression vector of any one of Embodiments 1 to 17, comprising two or more heterologous nucleotide sequence expressing a heterologous polypeptide.
   19. A viral particle comprising a viral expression vector of any one of Embodiments 1 to 18.
   20. A composition of the viral expression vector or viral particle of any one of Embodiments 1 to 19.
   21. The composition of Embodiment 20 further comprising an adjuvant.
   22. A method of expressing a heterologous polypeptide in a cell, the method comprising contacting the cell with a viral expression vector, viral particle, or composition of any one of Embodiments 1 to 21.
   23. A method of inducing an immune response in a subject to a heterologous polypeptide, the method comprising administering a viral expression vector, viral particle, or composition of any one of Embodiments 1 to 21 to the subject.
   24. The method of Embodiment 23, wherein the immune response comprises a humoral immune response and/or a cellular immune response.
   25. A method of expressing a heterologous polypeptide in a subject, the method comprising administering a viral expression vector, viral particle, or composition of any one of Embodiments 1 to 21 to the subject.
   26. A method of vaccinating a subject, the method comprising administering a viral expression vector, viral particle, or composition of any one of Embodiments 1 to 21 to the subject.
   27. The method of any one of Embodiments 23 to 26, wherein the viral expression vector, viral particle, or composition is administered intranasally, intramuscularly, topically, orally, or in ovo.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Evaluation of a New Viral Vaccine Vector in Mice and Rhesus Macaques:

### J Paramyxovirus (JPV)-Expressing HA of Influenza a Virus H5N1

To investigate JPV as a vaccine vector, the SH gene was replaced with the hemagglutinin (HA) gene from H5N1 (rJPVΔSH-H5), examined the immunogenicity of a single dose intranasal immunization of rJPVΔSH-H5 in mice and assessed its efficacy in mice against lethal H5N1 challenge. Also, the immunogenicity of intranasal vaccination of rJPVΔSH-H5 in rhesus macaques was evaluated and the humoral and cell-mediated immune responses assessed.

### RESULTS

### Generation and In Vitro Analysis of a Recombinant JPV Expressing HA

To generate a recombinant JPV expressing HA of H5N1 (rJPV-ΔSH-H5), the SH coding sequence in a full-length JPV plasmid was replaced with HA (FIG. 1A). This plasmid, together with three helper plasmids encoding N, P, and L proteins, and a plasmid encoding T7 RNA polymerase, were co-transfected into HEK293T cells and co-cultured with Vero cells as described previously (Li et al., 2013, J Virol; 87:12990-8). Vero cells were used to select a plaque-purified clone of the rJPV-ΔSH-H5 virus. After obtaining the rescued virus, PCR amplification of cDNA with JPV-specific primers was used to identify rJPV-ΔSH-H5 (FIG. 1). The full-length genome sequence of plaque-purified rJPV-ΔSH-H5 was confirmed by Sanger sequencing.

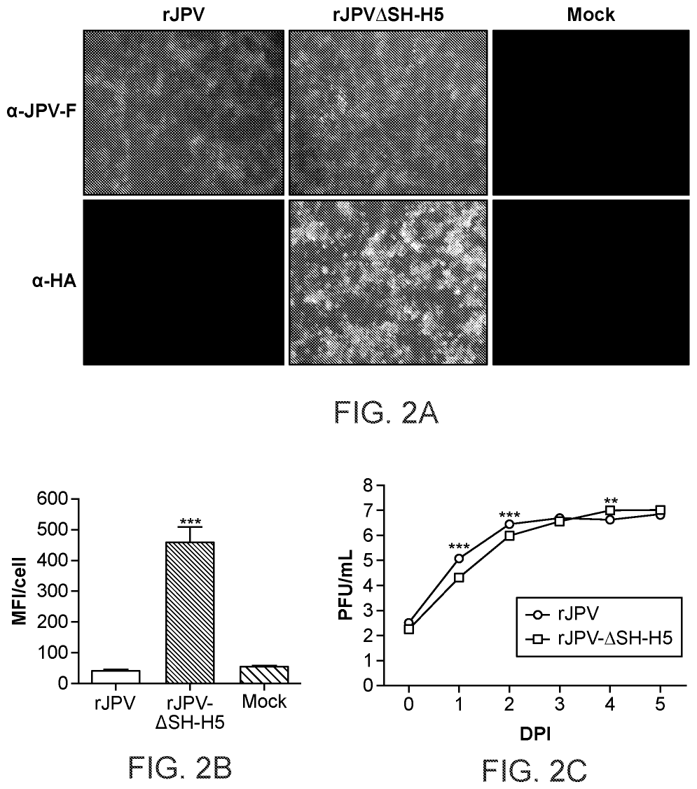

Expression of HA in rJPV-ΔSH-H5 infected Vero cells was confirmed using immunofluorescence assay with anti-mouse JPV F and H5N1 HA monoclonal antibodies (FIG. 2A). Quantification of H5N1 HA expression was determined using flow cytometry (FIG. 2B). To compare the growth kinetics of rJPV and rJPV-ΔSH-H5, Vero cells were infected with rJPV and rJPV-ΔSH-H5 at a multiplicity of infection (MOI) of 0.1. The medium was harvested at 24 hr time points, and viral titer in media were determined by plaque assay. Although a similar growth pattern was observed for rJPV and rJPV-ΔSH-H5, rJPV had a higher titer on 1 dpi and 2 dpi (FIG. 2C).

Determining the Immunogenicity and Efficacy of rJPV-ΔSH-H5 Against HPAI H5N1 Challenge in Mice

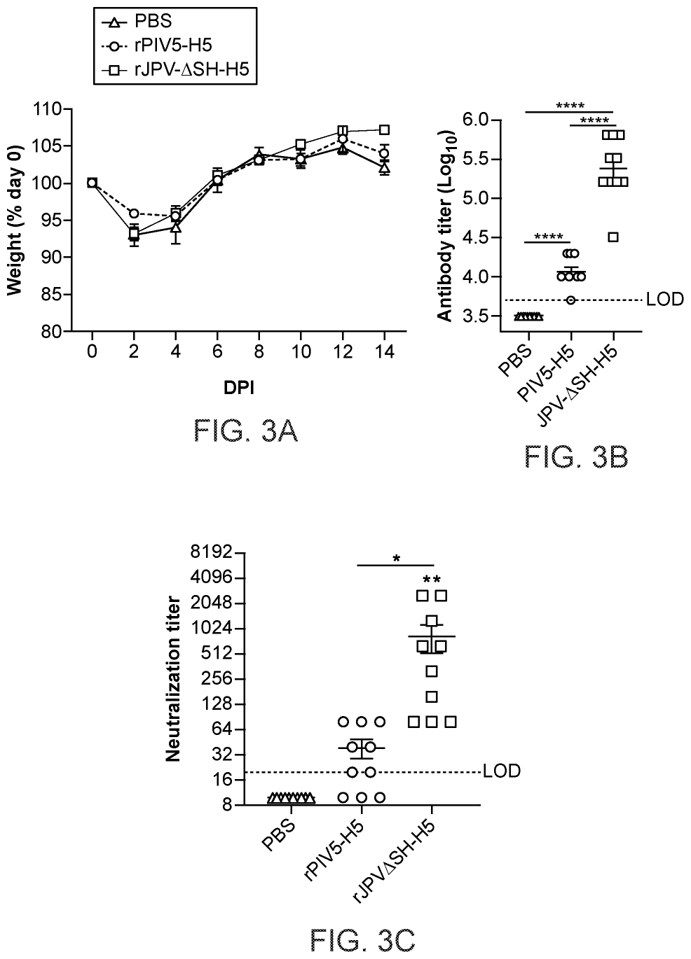

To investigate the immune response generated by rJPV-ΔSH-H5, mice were vaccinated with 100 μl of PBS or 1×10⁵ PFU each of rJPV-ΔSH-H5 or rPIV5-H5 intranasally. The weight of animals was monitored for 14 dpi. No difference in the body weight was observed in mice vaccinated with rJPV-ΔSH-H5 or rPIV5-H5 compared to the PBS group (FIG. 3A), indicating that the vaccine is attenuated in mice. Mice were bled at 28 dpi, and sera were used for ELISA to detect the H5-specific antibody response. rJPV-ΔSH-H5 induced a higher level of anti-H5-HA antibodies than rPIV5-H5 (FIG. 3B). Sera of mice immunized with rJPV-ΔSH-H5 generated antibodies that were able to neutralize PR8-H1N1 better than the sera of rPIV5-H5-immunized mice (FIG. 3C).

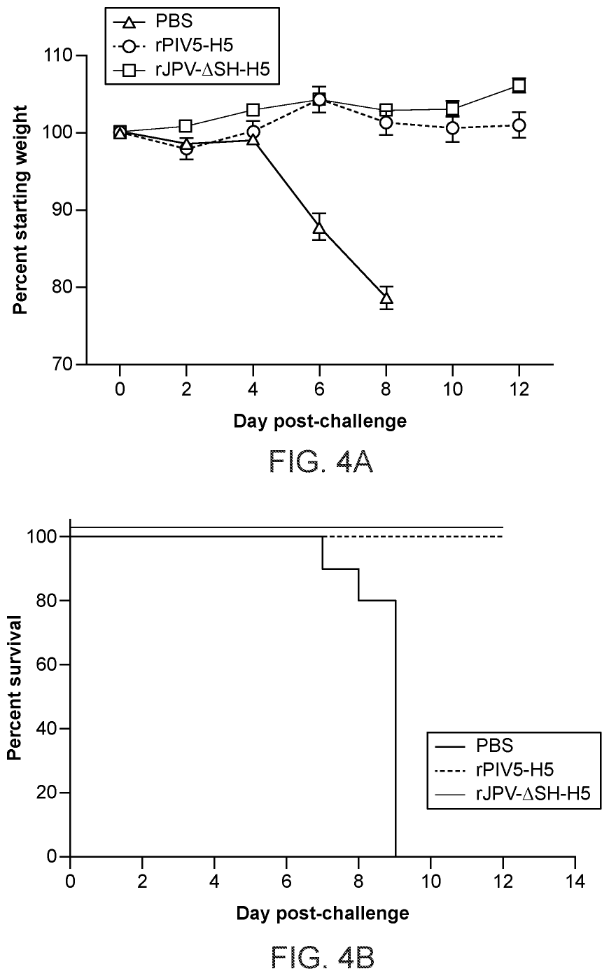

The efficacy of rJPV-ΔSH-H5 against HPAI H5N1 was examined in mice with A/Vietnam/1203/04 strain. Mice were vaccinated with 100 μl of PBS or 1×10⁵ PFU each of rJPV-ΔSH-H5 or rPIV5-H5 intranasally. At 73-day post-vaccination (d.p.v), mice were challenged with H5N1. All mice in the PBS group showed severe weight loss, and all animals were dead by day 9 after challenge (FIG. 4A). In contrast, all mice immunized with rJPV-ΔSH-H5 or rPIV5-H5 survived with mice immunized with rJPV-ΔSH-H5 experiencing no weight loss (FIG. 4B).

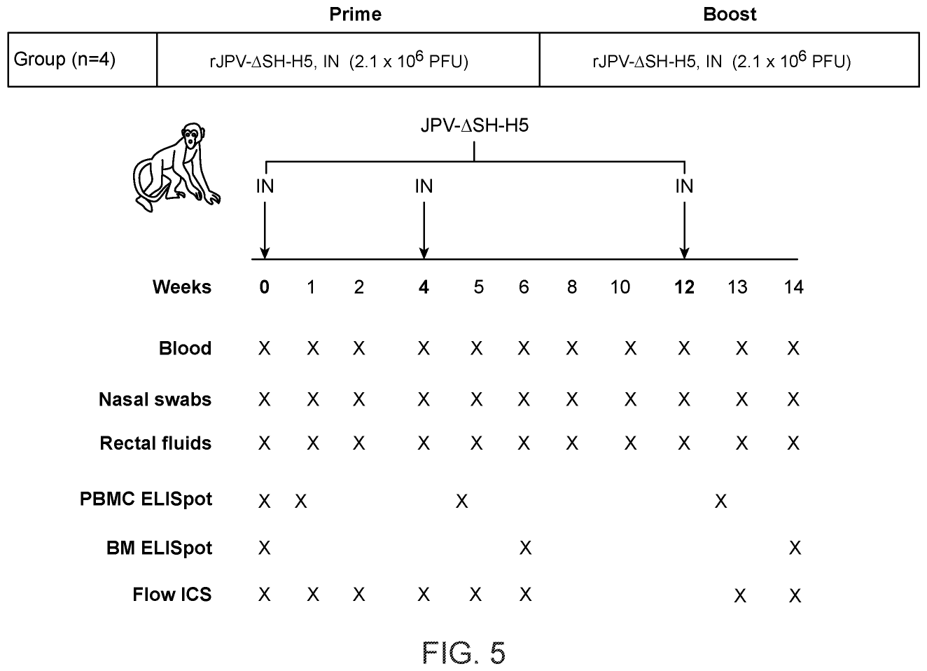

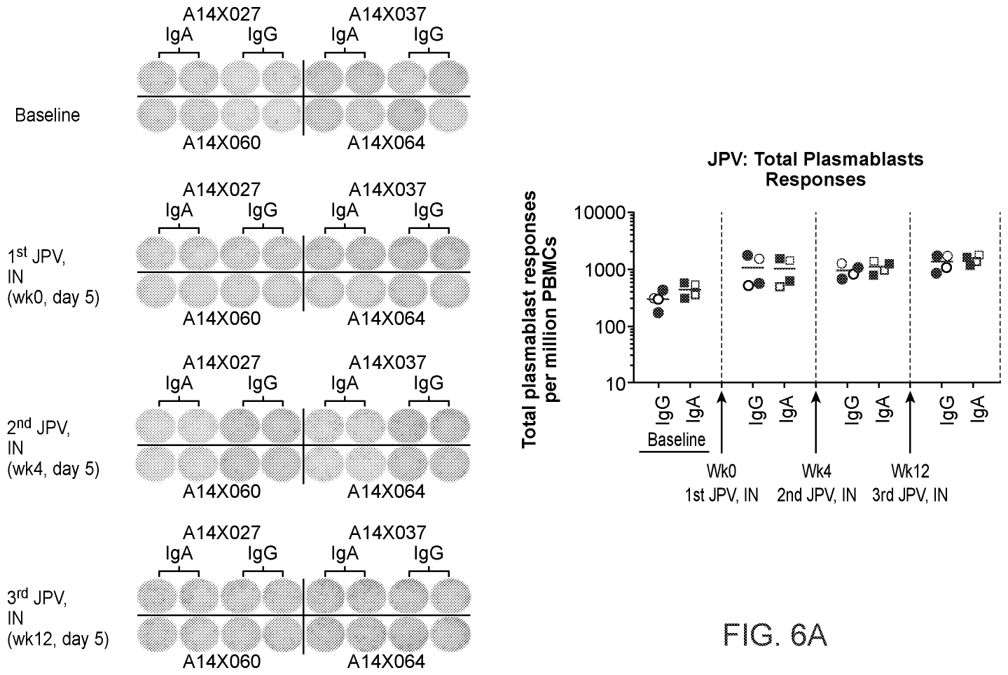

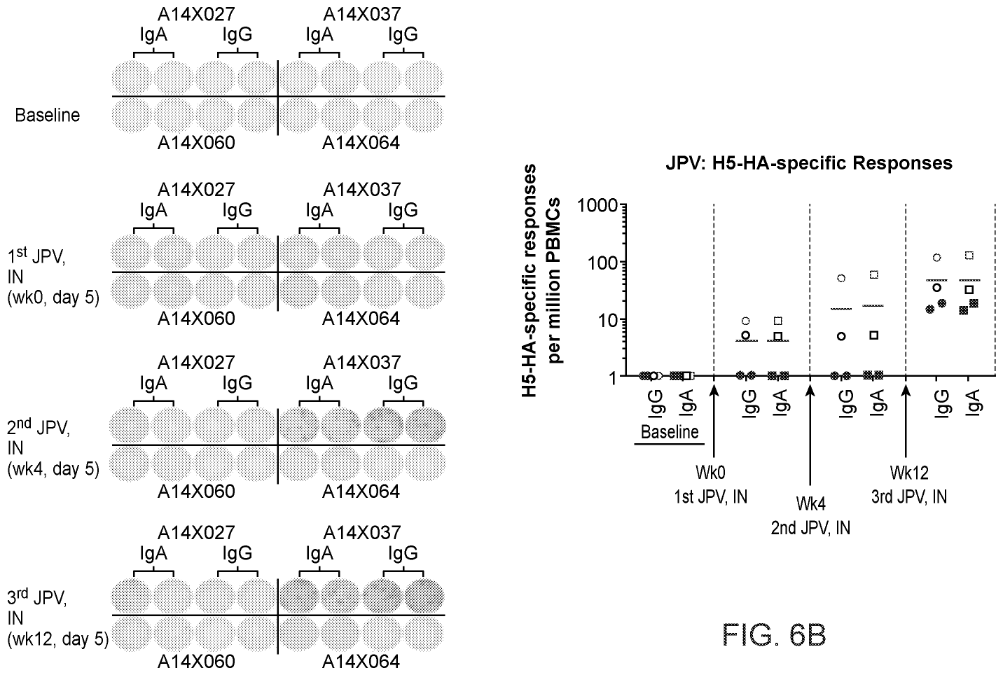

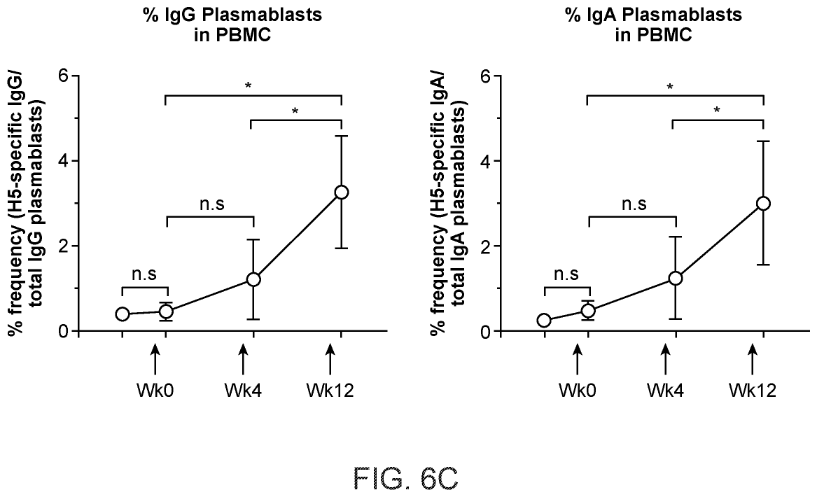

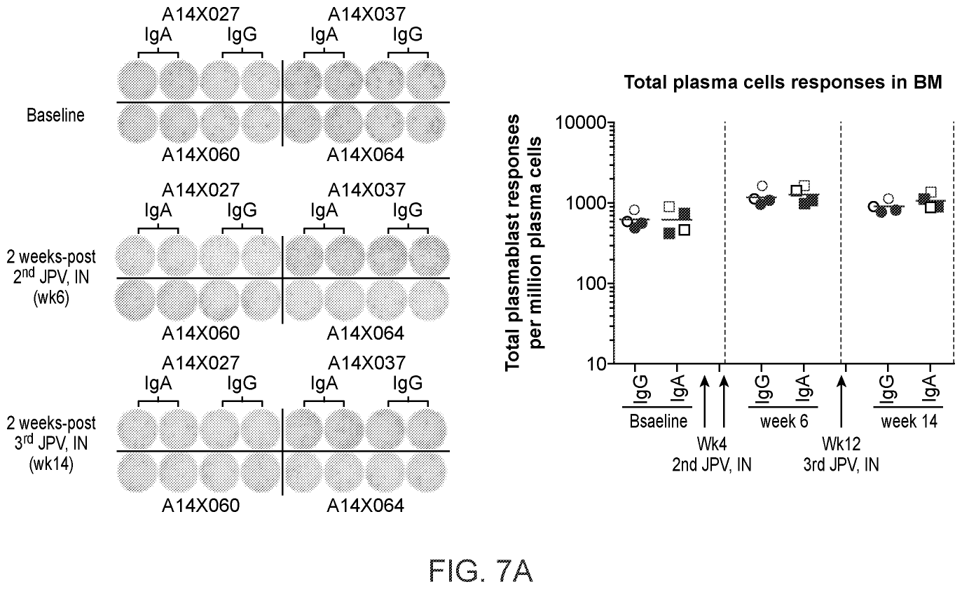

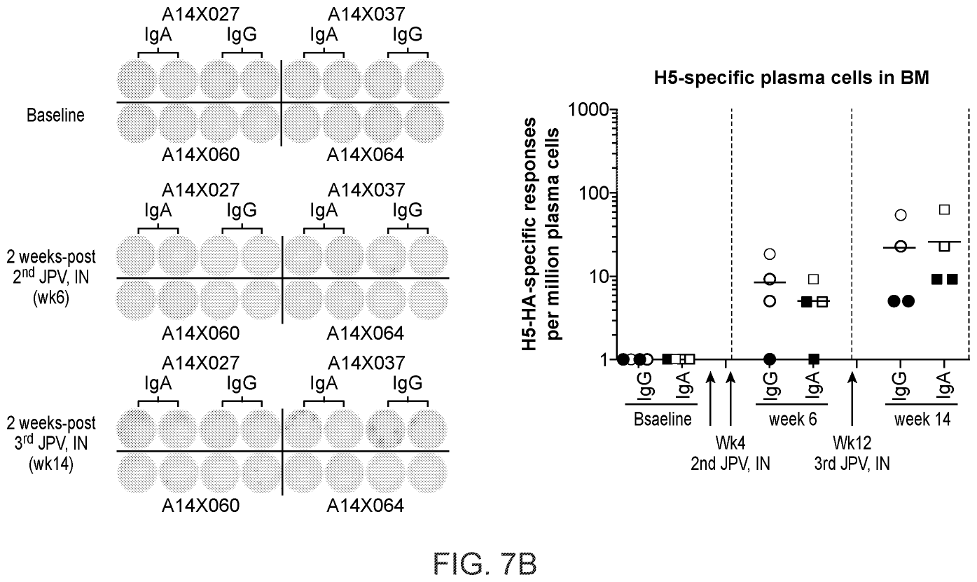

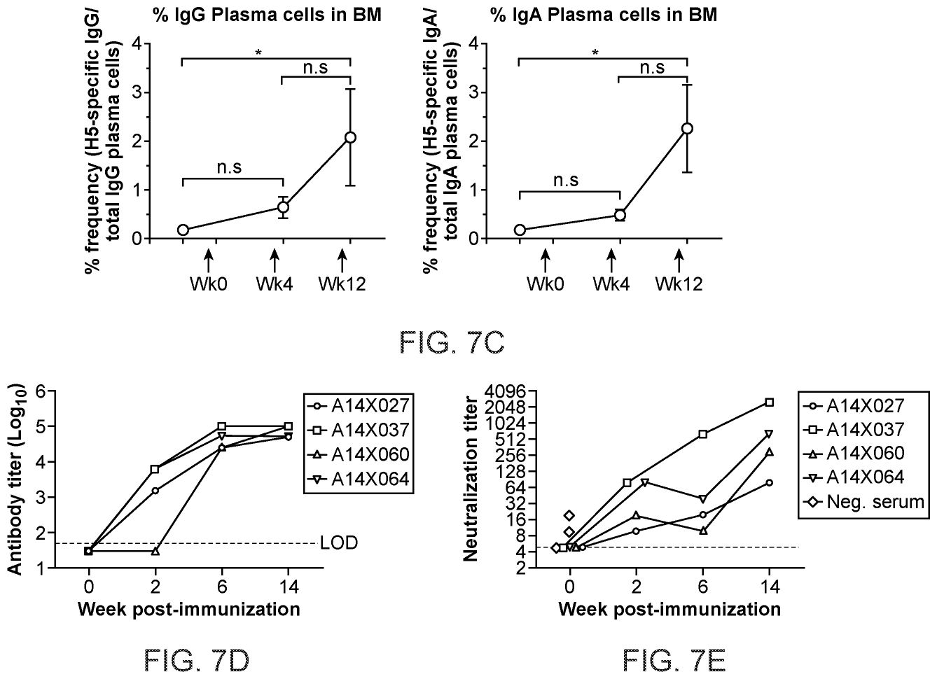

Determining the antibody response of rJPV-ΔSH-H5 vaccination in rhesus macaques Four rhesus macaques were intranasally immunized with 2.1×10⁶ PFU of rJPV-ΔSH-H5 on week 0, week 4, and week 12, as described in FIG. 5. Plaque assay with the rectal fluids and nasal swab of vaccinated animals did not detect the presence of live vaccine virus. Total and hemagglutinin (HA)-specific plasmablast responses were measured in blood using ELIspot assay. IgG and IgA responses of plasmablasts were analyzed 5 days after each immunization. Total plasmablast responses remained the same after prime and boost immunizations (FIG. 6A). However, H5 HA-specific IgG and IgA plasmablast responses increased, and the frequency of IgG and IgA plasmablasts significantly increased post-week 12 booster (FIGS. 6B and 6C). Similarly, plasma cell responses in bone marrow were measured 2 weeks after immunization. Total H5-specific plasma cell responses in bone marrow were similar after prime and boost immunizations (FIG. 7A), but H5 HA-specific IgG and IgA plasma cell responses and the frequency of IgG and IgA plasma cells in bone marrow significantly increased after week 12 booster (FIGS. 7B and 7C). Rhesus macaques were bled after prime and boost immunizations, and sera were used for ELISA to detect the H5-specific IgG antibody response. rJPV-ΔSH-H5 induced increased levels of anti-H5-HA antibodies following the prime and first boost with two of the macaques having their peak antibody titers at 6 weeks post-prime. Two of the four macaques had a slight increase in antibody titer following the second boost (FIG. 7D). All four macaques generated antibodies that were able to neutralize a PR8 CDC vaccine virus expressing H5 and N1. Interestingly, for all the macaques, neutralization titers increased following the second boost (FIG. 7E).

Cell-Mediated Immune Response of rJPV-ΔSH-H5 Immunization in Rhesus Macaques

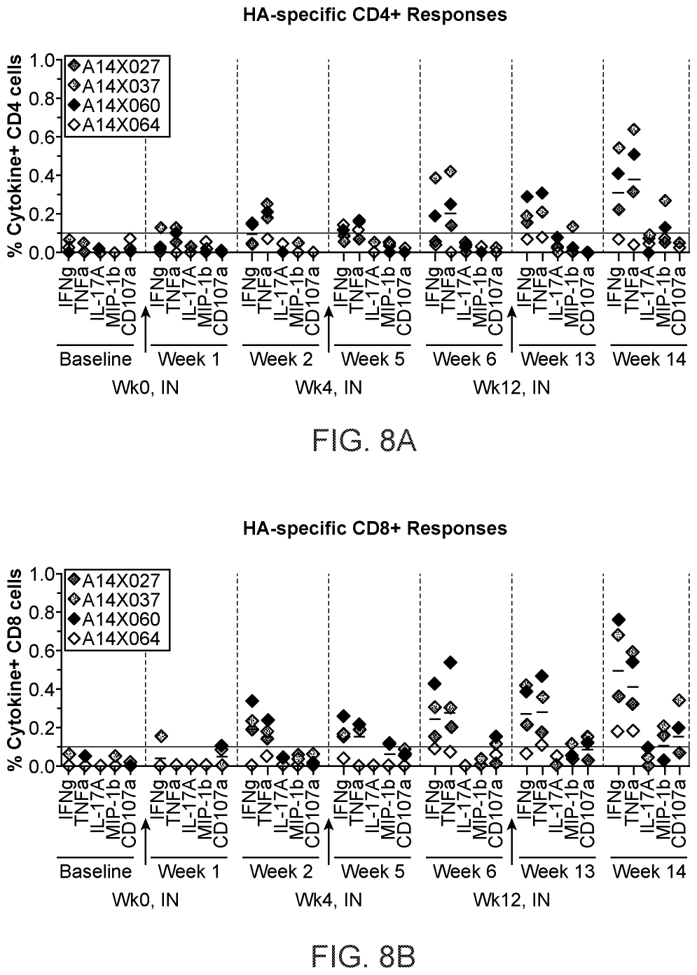

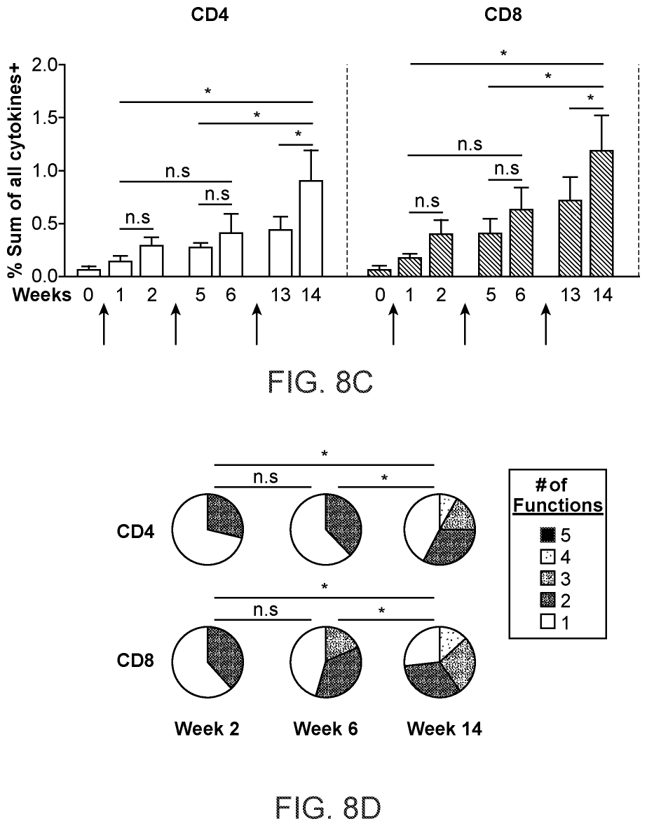

The induction of HA-specific CD4+ and CD8+ T cell responses in peripheral blood was determined after each prime and boost immunization via intracellular cytokine staining (ICS) for cells secreting IFN-γ, TNF-α, IL-17A, MIP-10 and CD107a following H5N1 HA peptides pool stimulation (FIGS. 8A and 8B). Following the initial immunization, CD4+ and CD8+ T cells specific for H5N1 HA were detectable at week 2, increased steadily at week 6, and markedly boosted at week 14 (FIGS. 8A and 8B). Among these cytokines detected, IFN-7 and TNF-α producing cells predominated the cellular response towards a Th-1 type effector phenotype, while IL-17A and MIP-10 responses were modest. CD107a, the degranulation marker was only detectable in CD8 T cells at week 14. The sum of the 5 cytokine responses for CD4 and CD8 is illustrated in FIG. 8C, illustrating the significant increase for both CD4+ and CD8+ T cell responses at week 14 (2 weeks post-boost immunization) (FIG. 8C). Next, the polyfunctional responses of HA-specific CD4+ and CD8+ T cells was evaluated, defined as cells that simultaneously produce multiple cytokines, a biomarker associated with more potent responses to vaccines. As shown in FIG. 8D, HA-specific CD4+ and CD8+ responses at week 14 were highly polyfunctional (P<0.05) compared to those responses at week 2 and week 6, respectively, with about 10% (CD4+) or 15% (CD8+) secreting four cytokines, though no response combining five cytokines were detected. In contrast, at week 2, only cells producing one or two cytokines were detected for both CD4 and CD8. CD4 responses were similar at week 6, but about 20% of CD8 cells produced three cytokines at week 6, though these differences did not reach statistical significance (FIG. 8D).

### DISCUSSION

Influenza causes 3-5 million severe cases annually, with 250,000-500,000 deaths globally. The reemergence of a pandemic H1N1 strain in 2009 (Neumann et al., 2009, Nature; 459:931-9) and the emergence of HPAI H5N1 and H7N9 influenza viruses (de Jong et al., 1997, Nature; 389:554; and Gao et al., 2013, N Engl J Med; 368:1888-1897) confirms that influenza is one of the most prominent global threats of this century. Although influenza vaccines have been available commercially since the 1940s, there are many limitations to these vaccines regarding availability and effectiveness. Currently most licensed influenza vaccines are produced in chicken eggs, which requires extensive time between the identification of vaccine strains and vaccine availability. Other limitations include lengthy regulatory approval procedures, limited worldwide vaccine availability, limited efficacy in elderly and unprimed populations, and lack of cross-reactivity requiring reimmunization during each season. The HPAI H5N1 virus was isolated for the first time from geese in Guangdong Province, China, in 1996 (Xu et al., 1999, Virology; 261:15-9). Since then, the virus has become endemic, causing a significant loss to the poultry industry with many human infections. Viral vectors such as adenovirus and vaccinia virus were used to develop H5N1 vaccines. However, pre-existing immunity and the requirement of multiple immunizations are clear limitations of these vaccines (Zhang, 2012, Viruses; 4:2711-2735; and Sebastian and Lambe, 2018, Vaccines; 6:e29). Currently, the only FDA-approved H5N1 vaccine must be administered multiple times at a high concentration to achieve a moderate level of efficacy compared to conventional influenza vaccines. Traditional vaccines against H5N1 utilizing the HA or NA of the virus are poorly immunogenic and suffer from production issues (Stephenson et al., 2004, Lancet Infect Dis; 4:499-509).

All NNSVs used for vaccine development replicates in the cytoplasm. As a result, similar to nucleoside-modified mRNA vaccines, NNSVs do not enter the nucleus and modifies the host DNA. mRNA-based vaccines which are often formulated with PEGylated lipid nanoparticles require an extensive cold chain for delivery. NNSVs are relatively more stable. Since it can replicate efficiently in the respiratory tract of primates, it is ideal to induce mucosal and systemic immune response. JPV has a large genome with eight transcriptional units. The deletion of multiple JPV genes has not affected the replication in vitro and in vivo. This feature allows the incorporation of large or multiple foreign genes into a JPV vaccine vector.

In this work, the rJPV-ASH backbone was used for developing an H5N1 vaccine candidate. rJPV-ΔSH-H5 grew similarly to rJPV in Vero cells and expressed the HA of H5N1. In vivo infection with rJPV-ΔSH-H5 or rPIV5-H5 did not cause weight loss compared to the PBS control group. A single dose of rJPV-ΔSH-H5 in mice induced HA-specific antibody responses and neutralization antibody titers against PR8 CDC vaccine virus expressing H5 and N1 (PR8-H5N1). Immunization with rJPV-ΔSH-H5 provided complete protection upon a lethal challenge with HPAI H5N1. Since JPV is a rodent virus, the production of HA-specific antibodies at high titers with rJPV-ΔSH-H5 compared to rPIV5-H5 may be due to the increased virus replication and transcription of JPV-encoded genes in the mouse respiratory tract. To study rJPV-ΔSH-H5 in eliciting humoral and cell-mediated immune responses in a non-human primate model, the effect of intranasal immunization with rJPV-ΔSH-H5 was tested in rhesus macaques. rJPV-ΔSH-H5 induced H5-specific IgG and IgA response in plasmablasts, antigen-specific memory response in bone marrow plasma cells, and H5-specific IgG antibodies in monkey sera. All four macaques generated neutralizing antibody titers against PR8-H5N1. Also, boosting monkeys with rJPV-ΔSH-H5 increased both H5-specific IgG response and neutralizing antibody response against PR8-H5N1. Successive immunizations with rJPV-ΔSH-H5 μlead to increased frequencies of long-lived HA-specific plasma cells in the bone marrow as well as increased HA-specific multifunctional CD4+ and CD8+ T cell responses, corresponding to not only quantitative but also qualitative desirable types of cellular responses. The development of a neutralizing immune response generated in rhesus macaques against the JPV vector was also observed, and increased plaque-reduction neutralization titers against WT JPV virus were observed with monkey sera. Interestingly, regardless of the vector immunity, booster vaccination of rJPV-ΔSH-H5 at the same dose still boosted both humoral and cell-mediated immune responses to HA in macaques.

### MATERIALS AND METHODS

### Cells

Human Embryonic Kidney 293T (HEK293T), Baby Hamster Kidney (BHK) cells, MDCK, and Vero cells (ATCC) were maintained in Dulbecco's modified Eagle's medium (DMEM) containing 10% fetal bovine serum (FBS), 100 IU/ml penicillin, and 100 μg/ml streptomycin. All cells were incubated at 37° C. in 5% CO₂. Cells infected with viruses were grown in DMEM containing 2% FBS. Vero cells were used to perform plaque assays of JPV, and BHK cells were used to perform plaque assays of PIV5.

### Influenza Virus

Highly pathogenic A/Vietnam/1203/2004 (H5N1) was propagated in the allantoic cavity of embryonated hen eggs at 37° C. for 24 h and were then aliquoted and stored at −80° C. Experiments involving HPAI were reviewed and approved by the institutional biosafety program at the University of Georgia and were conducted in enhanced biosafety level 3 (BSL3+) containment according to guidelines for the use of select agents approved by the CDC.

### Construction of Recombinant Plasmids

The construction of a recombinant JPV-BH plasmid with a PvuI restriction site at the N gene was previously described (Li et al., 2013, J Virol; 87:12990-8). By using standard molecular biology techniques, the ORF of the SH gene was replaced by an HA gene of H5N1. The construct lacking the SH gene and containing the HA gene was designated as a pJPV-ΔSH-H5 plasmid. A plasmid containing the H5N1 HA gene without the cleavage site was used as the DNA template for PCR amplification (Li et al., 2013, J Virol; 87:354-62).

### Virus Rescue and Sequencing

To generate viable recombinant JPV containing HA gene (rJPV-ΔSH-H5), a full-length pJPV-ΔSH-H5 plasmid, a plasmid expressing T7 polymerase (pT7P), and three plasmids encoding the N, P, and L proteins of JPV (pJPV-N, pJPV-P, and pJPV-L) were co-transfected into HEK293T cells at 95% confluency in a 6-cm plate with Jetprime (Polypus-Transfection, Inc., New York, NY). The amount of plasmids used were as follows: 5 μg of full-length pJPV-ΔSH-EGFP plasmid, 1 μg of pT7P, 1 μg of pJPV-N, 0.3 μg of pJPV-P, and 1.5 μg of pJPV-L. Two days post-transfection, 1/10^{th} of the HEK293T cells were co-cultured with 1×10⁶ Vero cells in a 10-cm plate. Seven days after coculture, media were centrifuged to remove the cell debris, and the supernatant was used for plaque assay in Vero cells to obtain single clones of recombinant JPV-ΔSH-H5. Vero cells were used to grow the plaque-purified virus. The full-length genomes of the plaque-purified rJPV-ΔSH-H5 virus isolates were sequenced. Total RNA of rJPV-ΔSH-H5-infected Vero cells were purified using the RNeasy minikit (Qiagen, Valencia, CA). cDNA was prepared by using random hexamers. PCR amplification of cDNA with primers MA12F and MA09R was used to identify rJPV-ΔSH-H5. DNA sequences were determined by an Applied Biosystems sequencer (ABI, Foster City, CA).

### Detection of Protein Expression

To confirm the rescue of JPV-ΔSH-H5, Vero cells were mock-infected or infected with rJPV or rJPV-ΔSH-H5. Vero cells were mock-infected or infected with rJPV or rJPV-ΔSH-H5 at an MOI of 0.1. At 2 d.p.i, cells were washed with phosphate-buffered saline (PBS) and fixed with 0.5% formaldehyde. The cells were permeabilized with 0.1% PBS-saponin solution and were incubated for 30 min with mouse monoclonal anti-F or -anti-H5N1 HA antibody at a 1:100 dilution (Genescript USA, Inc., Piscataway, NJ) and then fluorescein isothiocyanate (FITC)-labeled goat anti-mouse antibody was added to the cells. The cells were incubated for 30 min and were examined and photographed using a Nikon FXA fluorescence microscope.

To compare the expression of H5N1 HA in the virus-infected cells, Vero cells in the six-well plates were mock-infected or infected with rJPV or rJPV-ΔSH-H5 at an MOI of 5. The cells were collected at 2 d.p.i and fixed with 0.5% formaldehyde for 1 h. The fixed cells were resuspended in FBS-DMEM (50:50) and permeabilized with 70% ethanol overnight. The cells were washed once with PBS and then incubated with mouse anti-H5N1 HA monoclonal antibody in PBS-1% BSA (1:200) for 1 h at 4° C. The cells were stained with APC Goat anti-mouse IgG from Biolegend (1:500) for 1 h at 4° C. in the dark and then washed once with PBS-1% BSA. The fluorescence intensity was measured with a flow cytometer (Becton Dickinson LSR II).

### Growth Kinetics

Vero cells in 6-well plates were infected with rJPV or rJPV-ΔSH-H5 at an MOI of 0.1. The cells were then washed with PBS and maintained in DMEM-2% FBS. The medium was collected at 0, 24, 48, 96, and 120 hours post-infection (h.p.i). The titers were determined by plaque assay on Vero cells.

### Animals

6-week-old, female, BALB/c mice (Envigo) were used for the studies. Mice were infected with JPV and PIV5 in enhanced Biosafety Level 2 facilities in HEPA-filtered isolators. Mouse HPAI infections were performed in enhanced BSL3 facilities in HEPA-filtered isolators under the guidelines of the institutional biosafety program at the University of Georgia and for the select agents approved by the CDC. All animal experiments were performed in accordance with the national guidelines provided by “The Guide for Care and Use of Laboratory Animals” and the University of Georgia Institutional Animal Care and Use Committee (IACUC). The Institutional Animal Care and Use Committee (IACUC) of the University of Georgia approved all animal experiments. To test the immunogenicity of rJPV-ΔSH-H5, 6-week-old, female, BALB/c mice (Envigo) were infected with 100 μl of PBS or 1×10⁵ PFU each of rJPV-ΔSH-H5 or rPIV5-H5(11), intranasally. Plaque assays were performed for the back-titration of the virus inoculum used for the vaccination. The weight of the mice was monitored for up to 14 d.p.v. Twenty-eight d.p.v., the mice were bled for serum H5N1 HA-specific IgG titer. On 73 d.p.v., mice were anesthetized and inoculated intranasally with 10 50% lethal infectious doses (LD₅₀) A/Vietnam/1203/04 (27) diluted in 50 μl PBS. Mice were then monitored daily for morbidity and mortality with body weights measured every other day post-challenge.

Male Indian rhesus macaques (Macaca mulatta) were used in this study (body weight, 5-6 kg, averages 6.16±0.37 kg; age, 4 years, averages 4.14±0.02 year). Animals were sourced from the colonies of the New Iberia Research Center (NIRC) of the University of Louisiana at Lafayette and maintained in accordance with the rules and regulations of the Committee on the Care and Use of Laboratory Animal Resources. The study was approved by the University of Louisiana at Lafayette IACUC prior to its initiation. All animals were negative for SIV, simian T cell lymphotropic virus, and simian retrovirus. Rhesus macaques were immunized at weeks 0, 4, and 12 intranasally with the recombinant JPV-ΔSH-H5 containing 2.1×10⁶ PFU. Peripheral blood mononuclear cells (PBMCs) obtained throughout the immunization course were Ficoll purified and used for plasmablast ELISpot assay and intracellular cytokine staining (ICS). Bone marrow (BM) aspirates anticoagulated with heparin were Ficoll purified and used immediately for BM ELISpot assay. Serum samples were collected, aliquoted, and stored at −80° C. until used for neutralization assay. Nasal and rectal secretions were collected by Weck-Cel sponges and stored at −80° C. until use.

### ELISA

HA (H5N1 HA)-specific serum antibody titers were measured using an IgG enzyme-linked immunosorbent assay (ELISA). Immulon 2 HB 96-well microtiter plates (ThermoLabSystems) were coated with 1 μg/ml recombinant H5N1 HA protein and incubated at 4° C. overnight. Plates were then washed with KPL wash solution (KPL, Inc.), and the wells were blocked with 200 μl KPL wash solution with 5% nonfat dry milk and 0.5% BSA (blocking buffer) for 1 h at room temperature. Serial dilutions of serum samples were made (in blocking buffer), transferred to the coated plate, and incubated for 1 h. To detect bound serum antibodies, 100 μl of a 1:1000 dilution of Horseradish Peroxidase (HRP)-labeled goat anti-mouse IgG (KPL, Inc.) or HRP-labeled goat anti-monkey IgG (Abcam, Inc.) in blocking buffer was added per well and incubated for 1 h at room temperature. Plates were developed by adding 100 μl of Sureblue Reserve TMB Microwell peroxidase substrate (1-component), and the reaction was allowed to develop at room temperature. After 3-5 minutes, the reaction was terminated with 100 μl/well of 1N HCl. Optical density (OD) was measured at 450 nm on a Bio-Tek Powerwave XS plate reader. For the mouse serum, antibody titer was defined as the highest serum dilution at which the OD₄₅₀ was higher than the PBS average OD₄₅₀ plus two times the standard deviation. For the macaque serum, antibody titer was defined as the highest serum dilution at which the OD₄₅₀ was higher than the week 0 average OD₄₅₀ plus two times the standard deviation.

### PR8-H5N1 Neutralization Assay

A/Viet Nam/1203/2004 (H5N1) 6:2 on PR8 CDC vaccine strain (PR8-H5N1) was propagated in MDCK cells with Opti-MEM plus 100 IU/ml penicillin, 100 μg/ml streptomycin, and 2 ug/mL TPCK-trypsin. The virus was collected and titered via plaque assay on MDCK cells with a DMEM+5% FBS, 100 IU/ml penicillin, 100 μg/ml streptomycin, 2 ug/mL trypsin, and 1% agarose overlay. For the neutralization assay, serum was heat-inactivated at 56° C. for 45 minutes and serially diluted 2-fold in PBS. Each serially-diluted serum sample was incubated 1:1 with 50 PFU PR8-H5N1 at 37° C. for 1 hr. Following the incubation, the serum/PR8-H5N1 mixture was added onto 12-well MDCKs and incubated at 37° C. for 1 hr. The serum/PR8-H5N1 was removed, the cells were washed with DMEM, and an agarose overlay was added as described above. Plaques were counted 5 days later. The neutralization titer of each sample was defined as the highest dilution at which the plaque reduction was 50% or less.

### Antibody Secreting Cell ELISpot Assay

Total and antigen-specific plasmablasts in peripheral blood were quantified by ELISpot assay at day 5 following each immunization. Briefly, 96-well multiscreen HTS filter plates (Millipore) were coated overnight at 4° C. with 10 μg/ml of anti-monkey IgG or IgA (H&L) goat antibody (Rockland) or with 10 μg/ml of H5 protein (H5N1, A/Vietnam/1203/2004, Immune Tech) for enumeration of total or antigen-specific antibody-secreting cells (ASCs), respectively. Plates were washed and blocked for 2 h. Freshly isolated PBMCs were plated in serial 3-fold dilutions in duplicates and incubated overnight in a 5% CO₂ incubator at 37° C. Plates were washed and incubated with 1:1,000 diluted either anti-monkey IgG- or IgA-biotin conjugated antibodies (Rockland) for 1 h at 37° C. After washing, plates were incubated with 1:1,000 diluted horseradish peroxidase-conjugated Avidin D (Vector Labs) for 1 h at 37° C. and finally developed using AEC substrate kit (BD Biosciences). To stop the reaction, plates were washed extensively with water followed by air-drying. Spots were imaged and counted using the Immunospot ELISPOT Analyzer (Cellular Technology Limited). The number of spots specific for each Ig isotype was reported as the number of either total or antigen-specific spots per million PBMCs.

### Intracellular Cytokine Staining (ICS) Assay

The CD4+ and CD8+ T cell responses to H5N1 HA were quantified by ICS assay. Briefly, frozen PBMCs were thawed and rested overnight in complete 10% FCS RPMI medium. The next day, 2×10⁶ cells were stimulated with H5N1 HA peptide pools (BEI Resources, strain A/Vietnam/1203/2004) at a 1 μg/ml final concentration in the presence of anti-CD28 ECD (1 g/ml, clone CD28.2; Beckman Coulter), anti-CD49d (1 μg/ml, clone 9F10; BD Biosciences) and anti-CD107a FITC antibodies (1 μg/ml, clone eBioH4A3; eBioscience). Cells were cultured for 2 h before adding Brefeldin A (10 μg/ml, BD Biosciences) for an additional 4 h. An unstimulated control (dimethyl sulfoxide only) and positive control (PMA/Ionomycin) were included for each assay. After stimulation, cells were stained with the following antibodies: anti-CD3 Alexa700 (clone SP34-2; BD Biosciences), anti-CD4 BV605 (clone OKT4; BioLegend), anti-CD8 BV450 (clone RPA-T8, BD Biosciences), and anti-CD95 PE-Cy5 (clone DX2, BD Biosciences). An Aqua viability dye (Invitrogen) was added to the antibody cocktail to exclude dead cell background. Cells were washed, fixed and permeabilized using Fixation/Permeabilization buffer set (BD Biosciences), and incubated with anti-IFN-7 PE-Cy7 (clone B27; BD Biosciences), anti-TNF-α APC-Cy7 (clone Mab11; BioLegend), anti-IL-17A PE (clone eBio64CAP17; eBioscience), and anti-MIP-10 APC (clone FL34Z3L; eBioscience) antibodies. After washing, cells were resuspended in 2% formaldehyde and acquired on BD FACSAria Fusion. The net percentages of cytokine-secreting CD4+ and CD8+ cells were determined by subtracting the values with unstimulated samples using the FlowJo software (version 10.7, BD Biosciences). To analyze T cell polyfunctionality, the Boolean combinations and frequency of the cytokine positive cells were determined by FlowJo software and the Pestle and SPICE 6.0 software (see the worldwide web at niaid.github.io/spice/; Vaccine Research Center, NIAID, NIH). Student's t-test and permutation test were used for pie comparison between two groups.

### Statistical Analysis

Statistical analysis was performed using GraphPad Prism software (version 7). The Wilcoxon rank-sum test evaluated differences between the two groups. The one-way ANOVA test was used for comparisons across three or more groups. The distribution of the polyfunctional responses was analyzed by using Student's t-test and permutation comparison test for pie charts between groups. All statistical analyses were considered significant if they produced P values of <0.05.

This example has published as Abraham et al., “Evaluation of a New Viral Vaccine Vector in Mice and Rhesus Macaques: J Paramyxovirus Expressing Hemagglutinin of Influenza A Virus H5N1,” J Virol. 2021 Oct. 27; 95(22):e0132121. doi: 10.1128/JVI.01321-21. Epub 2021 Sep. 1, which is herein incorporated by reference in its entirety.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A viral expression vector comprising a J Paramyxovirus (JPV) genome comprising a heterologous nucleotide sequence expressing a heterologous polypeptide inserted within the JPV genome.

2. The viral expression vector of claim 1, wherein at least a portion of a JPV gene has been replaced with the heterologous nucleotide sequence.

3. The viral expression vector of claim 1, wherein the heterologous nucleotide sequence replaces at least a part of:
the N gene of the JPV genome;
the P gene of the JPV genome;
the M gene of the JPV genome;
the F gene of the JPV genome;
the SH gene of the JPV genome;
the TM gene of the JPV genome;
the G gene of the JPV genome;
the X gene of the JPV genome; and/or
the L gene of the JPV genome.

4. The viral expression vector of claim 1, wherein the heterologous nucleotide sequence replaces the SH gene nucleotide sequence.

5. The viral expression vector of claim 1, wherein the heterologous nucleotide sequence is inserted:
between the N and P genes of the JPV genome;
between the P and M genes of the JPV genome;
between the M and F genes of the JPV genome;
between the F and SH genes of the JPV genome;
between the SH and TM genes of the JPV genome;
between the TM and G genes of the JPV genome;
between the G and X genes of the JPV genome;
between the X and L genes of the JPV genome
within the N gene of the JPV genome;
within the P gene of the JPV genome;
within the M gene of the JPV genome;
within the F gene of the JPV genome;
within the SH gene of the JPV genome;
within the TM gene of the JPV genome;
within the G gene of the JPV genome;
within the X gene of the JPV genome; and/or
within the L gene of the JPV genome.

6. (canceled)

7. The viral expression vector of claim 1, wherein the JPV genome further comprises one or more mutations.

8. The viral expression vector of claim 1, wherein the heterologous polypeptide comprises an influenza hemagglutinin (HA), an influenza neuraminidase (NA), an influenza nucleocapsid protein (NP), influenza M1, influenza M2, influenza PA, influenza PB1, influenza PB2, influenza PB1-F2, influenza NS1 or influenza NS2.

9. The viral expression vector of claim 8, wherein the influenza comprises influenza A, influenza B, or influenza C virus.

10. The viral expression vector of claim 8, wherein the heterologous polypeptide comprises a hemagglutinin (HA) from influenza A virus strain subtype H1 to H18

11. The viral expression vector of claim 8, wherein the heterologous polypeptide comprises a hemagglutinin (HA) from influenza A virus strain H5N1, H3N2, or H1N1.

12. (canceled)

13. The viral expression vector of claim 8, wherein the heterologous polypeptide comprises an influenza neuraminidase (NA) from influenza type A subtype N1 to N10.

14. (canceled)

15. The viral expression vector of claim 1, wherein the heterologous polypeptide is derived from human immunodeficiency virus (HIV), parainfluenza virus 1, parainfluenza virus 2, parainfluenza virus 3, parainfluenza virus 4, human respiratory syncytial virus, bovine respiratory syncytial virus, human metapneumovirus, avian influenza, canine influenza, avian metapneumovirus, Nipah virus, Hendra virus, rabies virus, Ebola virus, porcine circovirus, porcine reproductive and respiratory syndrome virus, swine influenza virus, New Castle disease virus, mumps virus, measles virus, canine distemper virus, feline leukemia virus, human calicivirus, veterinary calicivirus, human norovirus, veterinary norovirus, rinderpest virus, and/or Mycobacterium tuberculosis.

16. The viral expression vector of claim 1, wherein the heterologous polypeptide is derived from a bacterium or a parasite.

17. The viral expression vector of claim 1, comprising two or more heterologous nucleotide sequence expressing a heterologous polypeptide.

18. A viral particle comprising a viral expression vector of claim 1.

19. A composition of the viral expression vector or viral particle of claim 1.

20. (canceled)

21. A method of expressing a heterologous polypeptide in a cell, the method comprising contacting the cell with a viral expression vector, viral particle, or composition of claim 1.

22. A method of inducing an immune response in a subject to a heterologous polypeptide, the method comprising administering a viral expression vector, viral particle, or composition of claim 1 to the subject.

23. (canceled)

24. A method of vaccinating a subject, the method comprising administering a viral expression vector, viral particle, or composition of claim 1 to the subject.

25. The method of claim 22, wherein the viral expression vector, viral particle, or composition is administered intranasally, intramuscularly, topically, orally, or in ovo.
